(19) United States
(12) Patent Application Publication
(10) Pub. No.: US 2006/0074353 A1
(43) Pub. Date: Apr. 6, 2006

(54) Glenoid instrumentation and associated method

(76) Inventors: Daren Lloyd Deffenbaugh, Winona Lake, IN (US); Thomas Scott Camino, Fort Wayne, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/951,022

(22) Filed: Sep. 27, 2004

(51) Int. Cl. A61B 5/103

(52) U.S. Cl. 600/587

(57) ABSTRACT

An instrument for measuring a defect in a glenoid fossa of a scapula is provided. The instrument includes a member for contact with the glenoid fossa and a probe. The probe is moveably associated with the member. The probe is operably associated with the defect for measuring the defect in the scapula.

### TECHNICAL FIELD OF THE INVENTION

The present invention relates generally to the field of orthopaedics, and more particularly, to an implant for use in arthroplasty.

### CROSS-REFERENCE TO RELATED APPLICATIONS

Cross reference is made to the following applications: DEP 5070 entitled “EXTENDED ARTICULATION PROSTHESIS ADAPTOR AND ASSOCIATED METHOD”, DEP 5072 entitled “GLENOID AUGMENT AND ASSOCIATED METHOD”, DEP 5304 entitled “INSTRUMENT FOR PREPARING AN IMPLANT SUPPORT SURFACE AND ASSOCIATED METHOD”, DEP 5306 entitled MODULAR GLENOID PROSTHESIS AND ASSOCIATED METHOD”, and DEP 5307 entitled “GLENOID INSTRUMENTATION AND ASSOCIATED METHOD”, filed concurrently herewith which are incorporated herein by reference.

### BACKGROUND OF THE INVENTION

During the lifetime of a patient, it may be necessary to perform a total shoulder replacement procedure on the patient as a result of, for example, disease or trauma. In a total shoulder replacement procedure, a humeral component having a head portion is utilized to replace the natural head portion of the arm bone or humerus. The humeral component typically has an elongated intramedullary stem which is utilized to secure the humeral component to the patient's humerus. In such a total shoulder replacement procedure, the natural glenoid surface of the scapula is resurfaced or otherwise replaced with a glenoid component that provides a bearing surface for the head portion of the humeral component.

As alluded to above, the need for a shoulder replacement procedure may be created by the presence of any one of a number of conditions. One such condition is the deterioration of the patient's scapula in the area proximate to the glenoid surface as a result of, for example, gleno-humeral arthritis. In such a condition, the erosion of the patient's scapula is generally observed posteriorly on the glenoid surface. Occasionally the erosion of the patient's scapula occurs anteriorly. Such erosion of the scapula renders treatment difficult, if not impossible, with a conventional glenoid prosthesis.

In order to treat a condition in which a portion of the scapula has been eroded, a number of glenoid prostheses have heretofore been designed. Such glenoid prostheses, known generally as augmented glenoid prostheses, have a posterior edge that is thicker than the corresponding anterior edge.

The design of the augmented glenoid component, however, has a number of associated drawbacks. For example, the relatively smooth, arcuate-shaped medial surface may over time lead to loosening of the augmented glenoid component, thereby potentially necessitating additional surgical procedures to replace or reseat the component. Further, due to the configuration of the medial surface, a relatively high shear load is created along the implant-to-bone interface when the component is implanted. The presence of a high shear load along the implant-to-bone interface tends to also cause loosening of the component 100 over a period of time. Post-operative loosening is the largest cause of failures of implanted glenoid components.

Another heretofore-designed augmented glenoid component has a single component plastic body. The thickness of the plastic body gradually increases from an anterior edge to a posterior edge thereof thereby creating a relatively smooth, arcuate-shaped medial surface from which a number of posts or pegs extend. The design of this augmented glenoid component, however, suffers from at least the same drawbacks as the glenoid component.

Prior attempts have been made to treat patients with posterior erosion of the glenoid. Many surgeons simply ream the glenoid surface to the proper orientation and implant the glenoid. Such a procedure leaves little supporting bone. Furthermore, because the little supporting bone is left there is almost no support bone available for a revision surgery if necessary.

Another common approach to treatment of posterior eroded glenoid is to ream the glenoid in a retroverted position. Although the glenoid is fully supported when utilizing such a ream approach, it is preferably loaded on the posterior edge. Such loading on the posterior edge can lead to loosing and failure of the glenoid component. A third option for treatment of glenoids with posterior erosion is a bone wedged graft. Such a bone wedged graft is technically difficult and has an inherent risk of failure.

More recently glenoid component have been developed that have a posterior augmentation. For example U.S. Pat. No. 6,699,289 to Iannotti and Williams, hereby incorporated by reference in its entirety has provided an option for treating glenoids with a posterior defect. Such glenoids with posterior augmentation are prepared utilizing a step cut method or removing more bone from the posterior portions of glenoid than from the anterior portion of the glenoid. These step cut glenoids require a proper characterization of the defect present in the natural glenoid.

There are currently no devices to provide the necessary information to the surgeon to aid in the implantation of these devices. One of the most common techniques surgeons use to verify the correct version is to place their finger on the anterior rim of glenoid fossa. It is has been reported that the correct angle between the anterior cortex and the plane of the glenoid is approximately 67 degrees. This information can be utilized to help a surgeon ensure that the glenoid implant is correctly implanted.

Referring now to FIG. 1 a prior art sizer disk 1 is shown in position on glenoid fossa 2. The sizer disk 1 is sized and has a shape to conform to a healthy glenoid fossa.

Referring now to FIG. 2 a diseased glenoid 3 is shown. The diseased glenoid 3 includes a portion of the natural glenoid fossa 4 which has eroded.

Referring now to FIG. 3 a naturally glenoid fossa shown which includes type C erosion or gradual erosion that is more pronounced posteriorly.

Referring now to FIG. 4 the prior art disk 1 is shown in position on posteriorly eroded natural glenoid fossa 3. As can be seen, the posterior erosion region 4 makes the use of the prior art sizer disk 1 problematic. The proper size or diameter of the prior art sized disk 1 cannot readily be determined due to the posterior erosion 4. Also, the fit of the prior art sizer disk 1 on the natural glenoid fossa 3 may be difficult to determine due to its shortened contact area.

### SUMMARY OF THE INVENTION

The present invention relates to novel instrumentation designed to give a surgeon a tool for deciding on the proper treatment for certain pathological conditions in the shoulder. Patients with posterior glenoid defects such as the type that includes bone loss need a special therapy to correct the defect. In order to properly and accurately treat the defect, it must be accurately characterized.

According to the present invention such characterization is accomplished with an instrument that tells the surgeon the appropriate size of the glenoid implant to be used. This instrument is augmented with a device to give the surgeon information about the size and depth of the posterior defect. This instrument or sizer disk may have a depth gauge. Such depth gauge may be a sliding rod type depth gauge and may be positioned on the rim of the sizer disk to give the surgeon information about the deepest portion of the defect. The sizer disk may have a wedge shape on the deepest portion of the defect corresponding to the specific glenoid implant. The sizer disk may also provide information to the surgeon on the correct version of the implant depending on the embodiments or combination of features.

The present invention describes several embodiments that are aspects capable, either alone or in concert, of giving the surgeon the necessary information for proper glenoid implantation. A first embodiment is a sizer disk with a protrusion on the anterior edge. This protrusion can be sufficiently long, yet narrow, to give the surgeon instant feedback on the version of the native glenoid or the reamed glenoid.

In some instances patients may have Type C erosion of the glenoid in which the glenoid fossa has been entirely eroded and the plane of the glenoid is apparently retroverted several degrees. This device will aide the surgeon in making that determination.

Another embodiment of the present invention may be in the form of a wedged shaped sizer disk that can be utilized on cases where there is Type C erosion. This disk will show the surgeon a more anatomical version, if not the correct version. Such a wedged shaped disk will allow the surgeon to size the implant necessary for such patients. This is necessary since due to the shape of the glenoid, as one moves more medially, the surface area of the glenoid fossa decreases. To make an attempt to return the glenoid to its anatomical version would result in excessive bone loss. The instruments of the present invention allow the surgeon to make an assessment of the proper size of the step cut for the glenoid that will be needed to correct the defect.

Another embodiment of the present invention is a sizer disk with a depth gage positioned on the posterior rim of sizer disk. The depth gauge may be in the form of a needle type depth gauge. This depth gauge allows the surgeon to properly size the glenoid to the existing bone and to measure the size of the step that will be required to correct the defect with the least amount of bone loss.

Since it is anticipated these devices will be used with a step cut glenoid system, they may be marked by etching, or other means, to determine the exact position of the central edge of the step augment. Such marking may aide the surgeon in assessing the glenoid for proper treatment.

According to one embodiment of the present invention, there is provided an instrument for measuring a defect in a glenoid fossa of a scapula. The instrument includes a member for contact with the glenoid fossa and a probe. The probe is moveably associated with the member. The probe is operably associated with the defect for measuring the defect in the scapula.

According to another embodiment of the present invention there is provided an instrument for measuring a defect in a glenoid fossa of a scapula. The instrument includes a body adapted to be secured to the scapula and an element. The element defines a surface of the element having a shape replicating that of a normal glenoid fossa. The element is securable to the body.

According to still another embodiment of the present invention there is provided a kit for measuring a defect in a worn glenoid fossa of a scapula. The kit includes a first sizing device defining a first surface for contact with the worn glenoid fossa and a second surface opposed to the first surface. The second surface has a shape conforming to a normal glenoid fossa. The first surface of the first sizing device is spaced from the second surface a first distance to represent a normal glenoid fossa. The kit also includes a second sizing device defining a first surface for contact with the worn glenoid fossa and a second surface opposed to the first surface. The second surface has a shape conforming to a normal glenoid fossa. The first surface is spaced from the second surface a second distance to represent a normal glenoid fossa. The second distance and the first distance being different from each other.

According to a further embodiment of the present invention, there is provided a method for providing arthroplasty on a glenoid fossa of a scapula. The method includes the step of providing a first glenoid component for attachment to the glenoid. The first glenoid component has a larger posterior dimension than the corresponding anterior dimension. The method also includes the step of providing a second glenoid component for attachment to the glenoid. The second glenoid component has a larger posterior dimension than the corresponding anterior dimension and has one dimension different from that of said first glenoid component. The method also includes the steps of providing a first sizing device corresponding to the first glenoid component and providing a second sizing device corresponding to the second glenoid component.

The method further includes the steps of placing the first sizing device against the glenoid fossa and placing the second sizing device against the glenoid fossa. The method also includes the step of determining which of the first glenoid component and the second glenoid component should be implanted onto the scapula, based on the placing of the one of the first sizing device and the second sizing device against the glenoid fossa. The method also includes the step of implanting the selected one of the first glenoid component and the second glenoid component.

The technical advantages of the present invention include the ability to accurately characterize a posterior defect. Such accurate characterizing of the posterior defect can be used to choose the appropriate glenoid implant for a posterior defect. For example, according to one aspect of the present invention an instrument for measuring a defect in a glenoid fossa of a scapula is provided. The instrument includes a member for contact with the glenoid fossa and a probe. The probe is removably associated with the member. The probe is operably associated with the defect for measuring the defect in the bone. Thus the present invention provides for an instrument which has an ability to accurately characterize a posterior defect.

The technical advantages of the present invention further include the ability of the instrument of the present invention to determine the size of a posterior defect on a glenoid. For example according to one aspect of the present invention an instrument for measuring a defect in a glenoid fossa of a scapula is provided. The instrument includes a member for contact with the glenoid fossa and a probe moveably associated with a member. The probe includes indicia located on the probe for indicating relative position of the probe with respect to the member. Thus the present invention provides for an instrument that can be used to determine the size of a posterior defect on a glenoid.

The technical advantages of the present invention include the ability of the instrument to be used to determine the shape of the posterior defect. For example, according to another aspect of the present invention a kit is provided for measuring a defect in worn glenoid fossa of a scapula. The kit includes a first sizing device defining a first surface and a second surface. The second surface is spaced a first distance from the first surface. The kit further includes a second sizing device having the first surface and a second surface spaced apart a second distance from the first surface. The first and second distances are different. Thus the present invention can be used to determine the shape of the posterior defect by placing the various sizing devices of the kit against the worn glenoid to determine the shape of the posterior defect.

The technical advantages of the present invention further include the ability to use the present invention to select one of plurality of posterior augment prostheses. For example according to another aspect of the present invention a kit is provided for measuring a defect in a worn glenoid fossa of a scapula. The kit includes a first sizing device and a second sizing device having a dimension different from the first sizing device. Each of the sizing devices may correspond to a particular augmented prosthesis. Thus the present invention can be used to select one of a plurality of posterior augmented prostheses.

The technical advantages of the present invention further include the ability of the present invention to be used to determine a specific measurement of the prosthesis needed. For example, according to one aspect of the present invention a instrument for measuring a defect in a glenoid fossa of a scapula is provided. The instrument includes a member for contact with the glenoid fossa and a probe moveably associated with the member for measuring the defect in the bone. The probe includes indicia thereon for indicating the relative position of the probe to the number. The indicia include marks, lines, alphabetic characters, or numbers in order to determine the specific measurement of the prosthesis. Thus the present invention provides for determining a specific measurement to determine the prosthesis needed.

The technical advantages of the present invention further include the ability to provide for a device for use in measuring Type C erosion of a glenoid cavity. For example, according an aspect of the present invention an instrument for measuring a defect in a glenoid fossa of a scapula is provided. The instrument includes a body adapted to be secured to the scapula and an element defining a surface thereof having a shape replicating that of a normal glenoid fossa. The body includes a protrusion for cooperation with an external cortical wall of the scapula. The protrusion may be adapted to secure the body to the scapula. By providing an instrument that is located against the exterior cortical wall of the scapula a glenoid cavity with Type C erosion or with an entire surface of the glenoid worn can be measured by locating the instrument on the exterior cortical wall of the glenoid.

Other technical advantages of the present invention will be readily apparent to one skilled in the art from the following figures, descriptions and claims.

### BRIEF DESCRIPTION OF THE DRAWINGS

FIG. 1 is an exploded plan view partially in cross section showing a natural glenoid fossa with a prior art sizer disk positioned over the glenoid fossa;

FIG. 2 is a plan view partially in cross section showing a natural glenoid fossa with a posterior erosion region;

FIG. 3 is a plan view partially in cross section showing a natural glenoid fossa with type C erosion;

FIG. 4 is an exploded plan view partially in cross section showing a natural glenoid fossa with a posterior erosion region and a prior art sizer disk positioned over the glenoid fossa;

FIG. 5 is a plan view partially in cross section showing a natural glenoid fossa with type C erosion region with an embodiment of a measurement instrument in the form of a disk with an external protrusion for cooperation with the scapula according to the present invention in cooperation with the glenoid fossa;

FIG. 5A is a partial plan view partially in cross section showing another embodiment of a instrument with a securement protrusion in accordance to the present invention in cooperation with a worn natural glenoid fossa;

FIG. 5B is a partial plan view partially in cross section showing another embodiment of a instrument with a separate securement pin in accordance to the present invention in cooperation with a worn natural glenoid fossa;

FIG. 6 is a plan view partially in cross section showing a natural glenoid fossa with type C erosion region with another embodiment of a measurement instrument in the form of a wedge according to the present invention in cooperation with the glenoid fossa;

FIG. 6A is a plan view of an instrument for use on a glenoid vault having posterior erosion according to yet another embodiment of the present invention;

FIG. 7 is a plan view partially in cross section showing a natural glenoid fossa with a posterior erosion region with yet another embodiment of a measurement instrument with a depth gage according to the present invention in cooperation with the glenoid fossa;

FIG. 7A is a partial plan view of the measurement instrument of FIG. 7 showing the contact area in greater detail;

FIG. 8 is an enlarged partial plan view partially in cross section showing the depth gage of the instrument of FIG. 7 in greater detail;

FIG. 9 is a plan view of a kit for use in performing shoulder arthroplasty in accordance to another embodiment of the present invention;

FIG. 9A is a plan view of another embodiment of the present invention in the form of kit of use with type C defects or for posterior erosion;

FIG. 10 is a flow chart for a method of performing shoulder arthroplasty in accordance to yet another embodiment of the present invention.

FIG. 11 is a plan view of a trial for use with type C erosion having an embedded sensor according to another embodiment of the present invention;

FIG. 12 is a plan view of a trial for use with posterior erosion having an embedded sensor according to another embodiment of the present invention.

FIG. 13 is a plan view of a gauge with a probe and an embedded sensor according to yet another embodiment of the present invention; and

FIG. 14 is a plan view of a trial with a sensor and a controller according to yet another embodiment of the present invention;

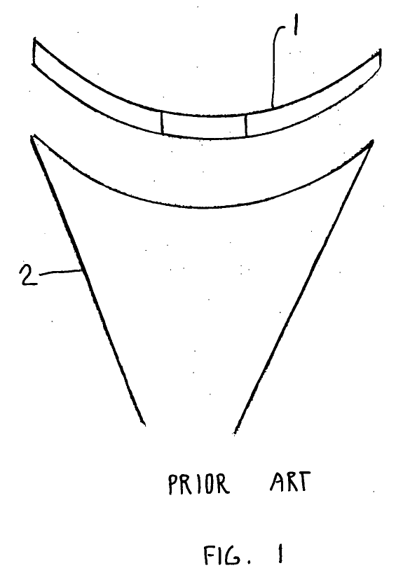

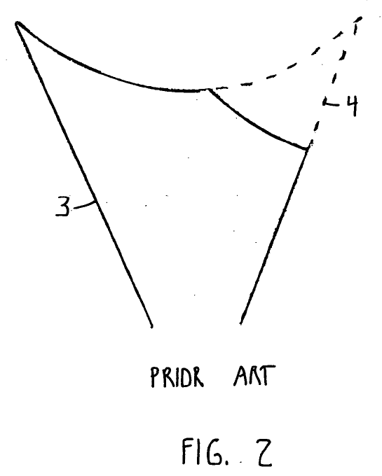

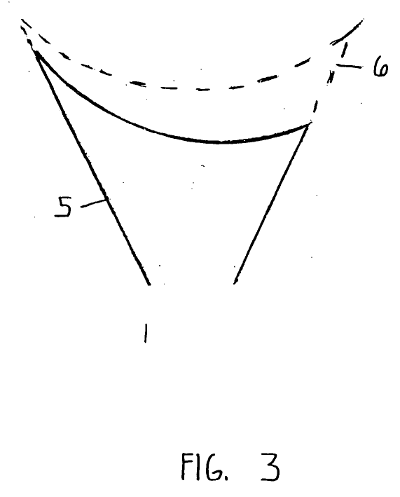

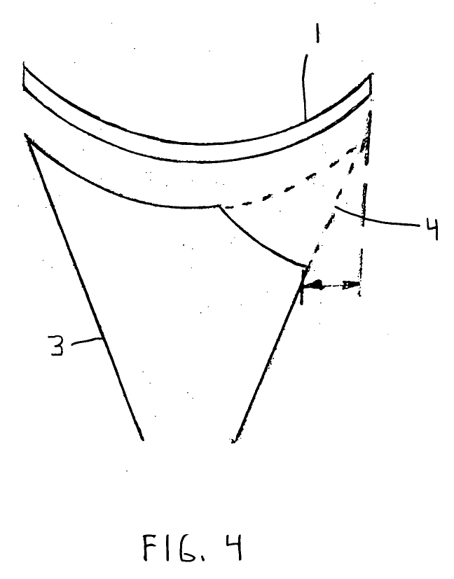

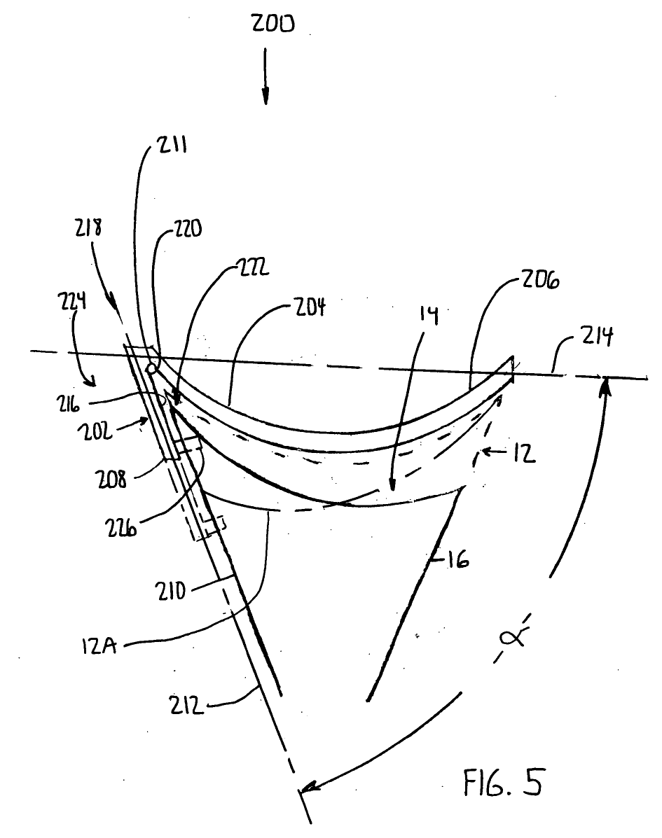

Corresponding reference characters indicate corresponding parts throughout the several views. Like reference characters tend to indicate like parts throughout the several views.

### DETAILED DESCRIPTION OF THE INVENTION

Embodiments of the present invention and the advantages thereof are best understood by referring to the following descriptions and drawings, wherein like numerals are used for like and corresponding parts of the drawings.

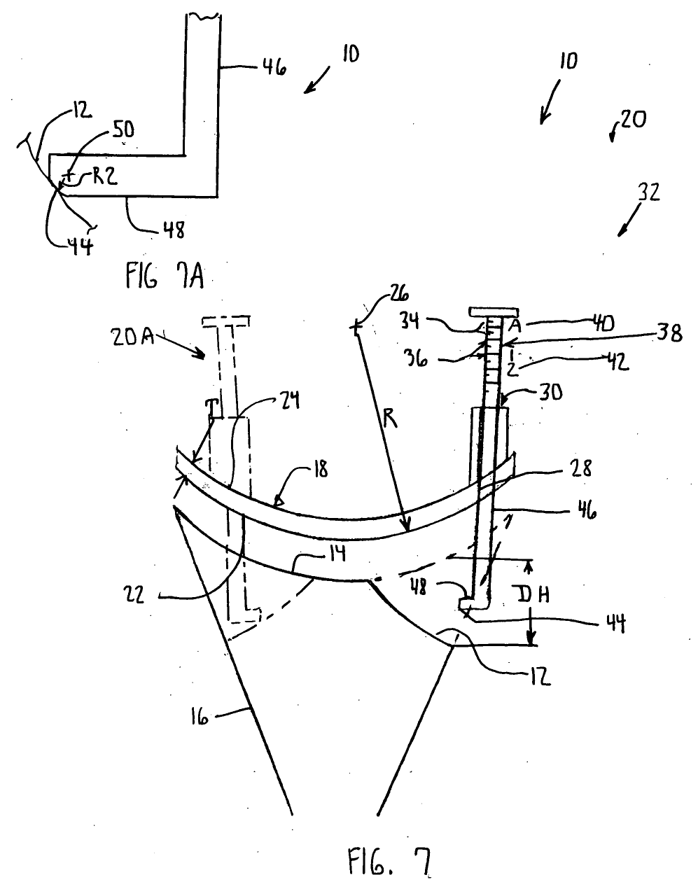

According to the present invention and referring now to FIG. 7 instrument 10 according to the present invention is shown. The instrument 10 is utilized for measuring a defect 12 as shown in phantom in a glenoid fossa 14 of a scapula 16. The instrument 10 includes a member 18 for contact with the glenoid fossa 14. The instrument 10 further includes a probe 20 moveably associated with the member 18. The probe 20 is operably associated with the defect 12 for measuring the defect 12 in the scapula 16.

As shown in FIG. 7 the member 18 may include a convex surface 22 for contact with glenoid fossa 14. For simplicity as shown in FIG. 7 the member 18 may be in the form of a curved plate having an opposed surface 24 opposed to the convex surface 22. The opposed surface may be concave. The opposed surface 24 and the convex surface 22 may define a thickness T there-between. The thickness may or may not be constant across the width of the device. The convex surface 22 may be, for example, a portion of a sphere and may be defined by a radius R extending from origin 26. The member 18, may as is shown in FIG. 7, include a wall 28 defining an opening 30 in the member 18. The probe 20 may be slideably fitted to the opening 30.

The probe 20 may include indicia 32 positioned on the probe 20. The indicia 32 may be utilized for indicating the relative position of the probe 20 with respect to the member 18.

The indicia 32 may be in any form that can be used to determine the position of the probe 20 with respect to the member 18. For example the indicia 32 may be in the form of marks 34 or lines 36. For example, parallel spaced apart lines 36 may be positioned along the probe 20. For example, marks 34 may be positioned alternatively between the lines 36. Further, the indicia 32 may also include characters 38. For example the character 38 may be in the form of alphabetic characters 40 or numeric characters 42. Also, the indicia 32 may also include colors or alternatively dark and light markings.

The probe 20 may include a contact area 44 for contact with the defect 12. The probe 20 may include a stem 46 which may include the contact area 44 and which may extend downwardly from the convex surface 22 of the member 18. The contact area 44 may be positioned on an arm or extension 48 extending from the stem 46. For simplicity the stem 46 and the opening 30 may be circular. Alternatively the stem 46 and the mating opening 30 may have a noncircular cross section. For example the stem 46 may have a rectangular, triangular, or a stem cross-section with a flat. A non-uniform cross-section for the stem 46 may serve to keep the arm or extension 48 in the proper angular position to contact the defect 12.

The instrument 10 may alternatively include a probe 20A as shown in phantom for measuring anterior erosion.

Referring now to FIG. 7A the contact area 44 of the arm 48 of the stem 46 of the probe 20 shown in greater detail. The contact area 44 of the probe 20 preferably conforms to the shape of the defect 12. For example, as is shown in FIG. 7A the contact area 44 may be generally arcuate. For example, the contact area 44 may be defined by a radius R2 extending from origin 50.

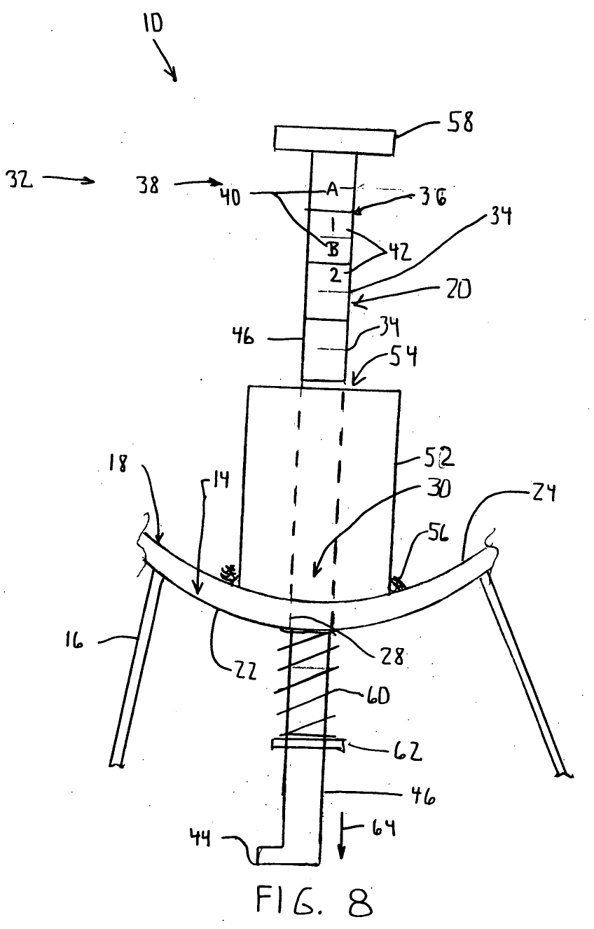

Referring now to FIG. 8 the probe 20 is shown in greater detail. As shown in FIG. 8 the instrument 10 may further include a bushing 52 secured to the member 18. The bushing 52 is utilized to provide a stable and accurate sliding movement of the probe 20. The bushing 52 includes a central opening 54 for the stem 46 of the probe 20 to slideably fit therewith. The bushing 52 may be secured to the member 18 in any fashion, for example, by a weldment 56.

The probe 20 may include a head 58 to prevent the probe 20 from moving downward out of the bushing 52. The probe 20 may also include an urging device in the form of, for example, a spring 60 which may be positioned over the stem 46. The spring 60 may be positioned between the member 18 and, for example, a stop 62. The spring 60 may be adapted to urge the stem 46 downward in the direction of arrow 64 to assure that the contact area 44 of the probe 20 is in contact with the defect 12 of the glenoid fossa 14.

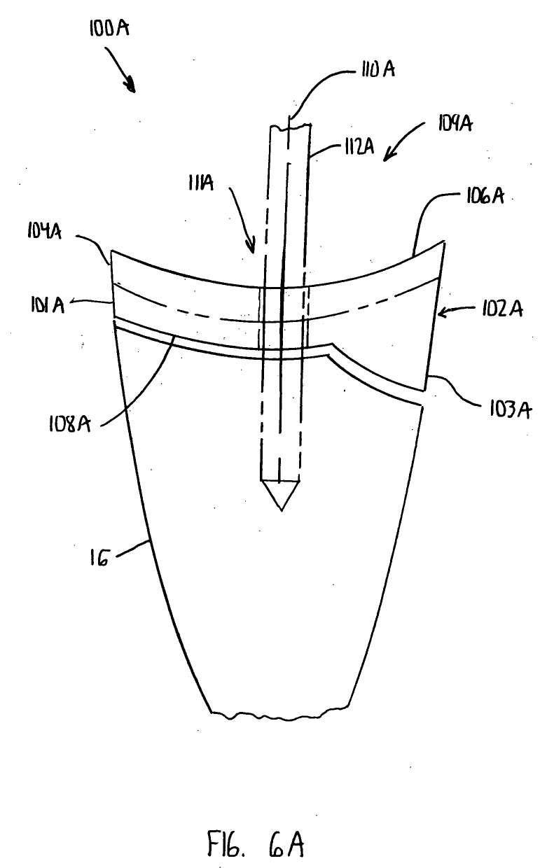

Referring now to FIG. 6 another embodiment of the present invention is shown as instrument 100. Instrument 100 is utilized for measuring a defect 12 in a glenoid fossa 14 of scapula 16. The instrument 100 includes a body 102 adapted to be secured to the scapula 16. The instrument 100 also includes an element 104 defining a surface 106 of the element 104 having a shape replicating that of a normal glenoid fossa. The element 104 is securable to the body 102. The instrument of the present invention may alternatively be designed for anterior erosion as shown as instrument 100A as shown in phantom.

The body 102 may be secured to the glenoid fossa 14 of the scapula 16 in any suitable manner for example as in shown in FIG. 6 the body 102 may include a convex surface 108 for contact with the glenoid fossa 14. The body 102 may be secured to the glenoid fossa 14 by for example merely holding the instrument 100 against the scapula 16 or by use of fasteners (not shown) such as pins, screws, clamps or the like. The element 104 may be secured to the body 102 in any suitable manner for example by screws, pins or other fasteners or may be molded or welded to the body 102. Alternatively, as is shown in FIG. 6 the element 104 and the body 102 may be integral with each other.

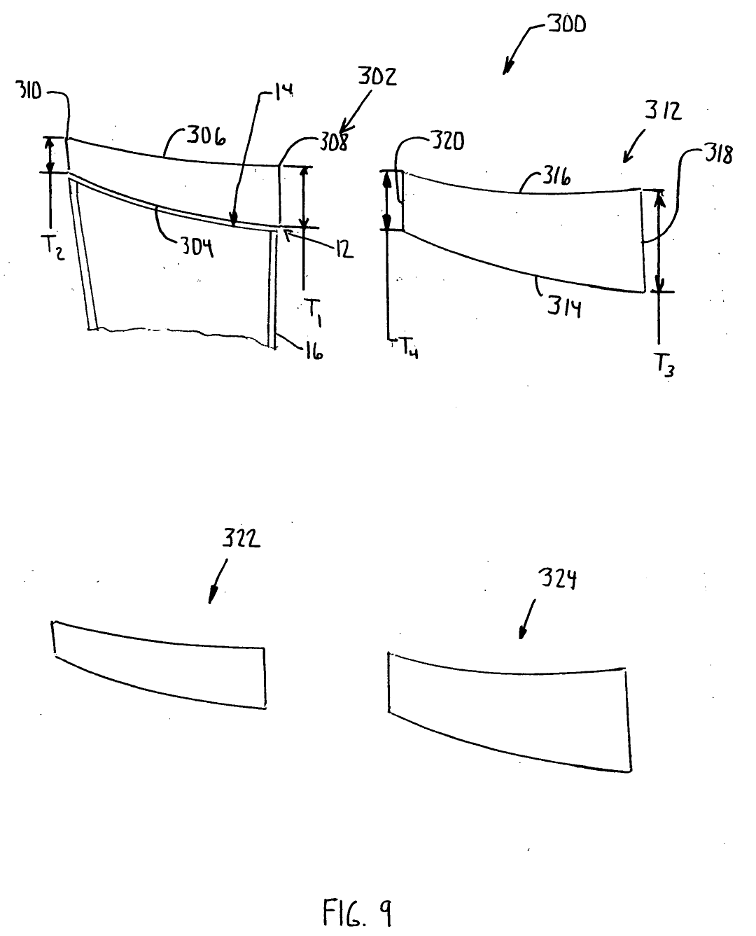

Preferably and as is shown in FIG. 6, the instrument 100 includes a pin guide 109 to assist in marking the axis of reconstruction anatomically. The pin guide 109, as shown in FIG. 9 may be in the form of an opening 111 which is formed in body 102 of the instrument 100. The opening 111 defines a reconstructive axis 110. The axis 110 is as is shown in FIG. 6, preferably normal or perpendicular to the articulating surface 106. The reconstructive axis 110 is preferably positioned centrally in glenoid vault 16. The opening 111 is sized to slideably receive pin 112. The opening 111 may serve as a drill guide or a guide to direct insertion of the pin 112 into the glenoid vault 16. The pin 112 provides for an anatomical axis of reconstruction for reconstructing the glenoid fossa.

Referring now to FIG. 6A, another embodiment of the present invention is shown as instrument 100A. The instrument 100A is similar to the instrument 100 of FIG. 6 but is used on a glenoid vault 16A having posterior erosion.

For example and as is shown in FIG. 6A, the instrument 100A includes a body 102A including posterior protrusion 103A to accommodate the posterior void of the glenoid vault as shown in phantom.

The body 102A may be integral or may include a base 101A defining support surface 108A. The body 102A may further include a protrusion 104A extending from the base 101A. The element 104A may define the articulating surface 106A.

The instrument 100A preferably and is shown in FIG. 6A, includes a pin guide 109A similar to the pin guide 109 of FIG. 6.

The pin guide 109A may, as is shown in FIG. 6A, be in the form of an opening 111A formed in the body 102A of the instrument 10A. The opening 111A may define reconstructive axis 110A. Preferably and is as shown in FIG. 6A, the reconstructive axis 110A is preferably perpendicular or normal to the concave surface 106A of the instrument 100A. The reconstructive axis 110A preferably is positioned centrally in the glenoid vault 16.

A pin 112A is slideably fitted in the opening 111A. The pin 112A may be a self-drilling and a self-tapping pin which may inserted into the glenoid vault 16A when the instrument 100A is in position. The pin 112A may be utilized to assist in the forming of a resurface glenoid fossa.

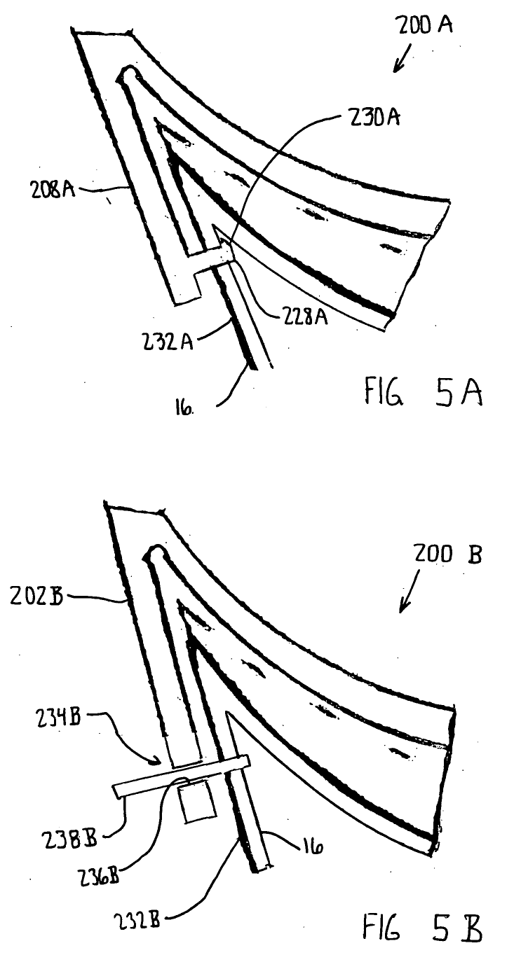

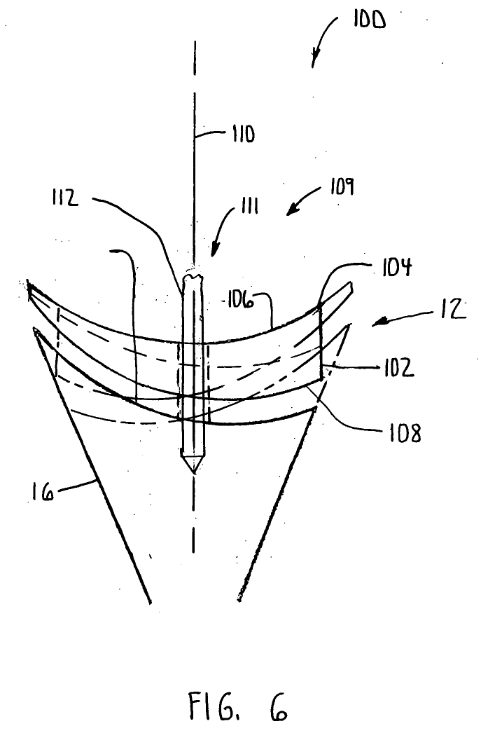

According to the present invention and referring now to FIG. 5 another embodiment of the present invention is shown as instrument 200. The instrument 200 is utilized for measuring a defect 12 in the glenoid fossa 14 of a scapula 16. The instrument 200 includes a body 202 adapted to be secured to the scapula 16. The instrument 200 further includes an element 204 defining a surface 206 of the element 204 having a shape replicating that of a normal glenoid fossa. The element 204 is securable to the body 202. It should be appreciated that the instrument 200 may be used with an anterior defect 12A as shown in phantom.

While the element 204 may be secured to the body 202 in any suitable fashion, for example by screws, pins or by welding, the body 202, as shown in FIG. 5, may be integral with the element 204. For example as shown in FIG. 5 the body may include a protrusion 208 for cooperation with an external cortical wall 210 of the scapula 16. The protrusion 208 is adapted to secure the body 202 to the scapula 16. As shown in FIG. 5 the protrusion 208 extends from an end-211 of the element 204 and, as is shown in FIG. 5, may be integral with the body 202.

As shown in FIG. 5 the body 202 may define a longitudinal axis 212 of the body 202. The element 204 may define a longitudinal axis 214 of the element 204. The longitudinal axis 212 of the body 202 and the longitudinal axis 214 of the element 204 may define an included angle α. The angle α may be any angle that serves to present the surface 206 of the element 204 in a position that may replicate that of a normal glenoid fossa. For example, the included angle α may be from 30-100 degrees and may be for example 50-90 degrees and may be around 70 degrees.

The instrument 200 may be secured to the scapula 16 in any suitable manner. For example, as shown in FIG. 5 the protrusion 208 of the body 202 of the instrument 200 may include a support face 216 against which the body 202 of the instrument 200 rests. The instrument 200 is positioned in the direction of arrow 218 until inner corner 220 of the instrument 200 rests against edge 222 of the glenoid fossa 14 and the instrument 200 is rotated in the direction of arrow 224 to assure that the support face 216 of the body 202 is secure against the scapula 16. The instrument 203 may be held manually in this position.

Alternatively and is shown in FIG. 5 in phantom the body 202 of the instrument 200 may include a pin 226 which may extend from the support face 216 of the body 208. The pin 226 may engage the scapula 16 to secure the instrument 200 in place.

Referring now to FIG. 5A, an alternate embodiment of the present invention is shown as instrument 200A. Instrument 200A similar to instrument 200 of FIG. 5 except that instrument 200A includes a protrusion 228A including barb 230A located on the protrusion 228A. The protrusion 228A preferably pierces through cortical wall 232A of the scapula 16 and the barb 230A serves to keep the protrusion 208A against the scapula 16.

Referring now to FIG. 5B another embodiment of the present invention is shown as instrument 200B instrument 200B is similar to instrument 200 of FIG. 5 except that the instrument 200B includes an opening 234B formed in body 202B of the instrument 200B. The opening 234B is defined by an internal wall 236B to which a pin 238B slideably fits. The pin 238B is used to engage with the cortical wall 232B of the scapula 16.

Referring now to FIG. 9, another embodiment of the present invention is shown as kit 300. The kit 300 is utilized for measuring a defect 12 in a worn glenoid fossa 14 of a scapula 16. The kit 300 includes a first sizing device 302 defining a first surface or support surface 304 for contact with the worn glenoid fossa 14. The first sizing device 302 also defines a second surface or articulating surface 306 opposed to the first surface 304. The second surface or articulating surface 306 has a shape conforming to a normal glenoid fossa. The first surface or support surface 304 is spaced from the second surface or articulating surface 306 represent a normal glenoid fossa. As shown in FIG. 9 the first sizing device 302 may have a different distance between the support surface 304 and the articulating surface 306 along the length of the support surface and articulating surface 304 and 306, respectively. For example, as shown in FIG. 9, the support surface 304 and the articulating surface 306 may be separated a distance T1 at the first end 308 of the first sizing device 302 and spaced apart a distance T2 at a second end 310 of the first sizing device 302.

Kit 300 may further include a second sizing device 312. The second sizing device 312 defines a first surface or support surface 314. The support surface 314 is utilized for contact with the worn glenoid fossa 14. The second sizing device 312 further defines a second surface 316 in the form of an articulating surface. The second surface or articulating surface 316 has a shape conforming to a normal glenoid fossa. The second surface 316 is opposed to the first surface 314. The second surface 316 is separated from the first surface 314 a distance T3 at a first end 318 of the second sizing device 312 and is separated a distance T4 at a second end 320 of the second sizing device 312. As shown in FIG. 9 the thickness T4 is different than the thickness T2 and the thickness T3 is different than the thickness T1. It should be appreciated either thicknesses T1 and T3 may be equal or thickness T2 and thickness T4 may be equal and provide for a difference in the first sizing device 302 from the second sizing device 312.

The kit 300 may as shown in FIG. 9 further include a first glenoid implant 322 corresponding to the first sizing device 302. Similarly the kit 300 may further include a second glenoid implant 324 corresponding to the second sizing device 312. The first sizing device 302 and the first glenoid implant 322 may, as shown in FIG. 9, have identical dimensions. Similarly, the second sizing device 312 and the second glenoid implant 324 may likewise have identical dimensions. Since the sizing devices and the implants have respective, identical dimensions, the sizing devices 302 and 312 may be utilized to determine which glenoid implant is proper for a particular worn glenoid. It should be appreciated that the kit 300, may in addition to the two sizing devices and two glenoid implants of FIG. 9, include additional sizing devices and glenoid implants to provide for more options for the surgeon.

According to the present invention and referring now to FIG. 9A, yet another embodiment of the present invention is shown as kit 300A. Kit 300A is similar to the kit 300 of FIG. 9 except that the kit 300A is intended for use with type C defects or for posterior erosion.

For example, and is shown in FIG. 9A, the kit 300A includes a first sizing device 302A. The first sizing device 302A includes a support surface 304A and an opposed articulating surface 306A. The first sizing device 302A includes a posterior protrusion 303A sized to accommodate a particular extent of posterior erosion on a patient.

Preferably and is shown in FIG. 9A, the sizing device 302A includes a pin guide 309A. The pin guide 309A may, as is shown in FIG. 9A, be in the form of an opening 311A formed in the first sizing device 302A. The opening 311A may define a reconstructive axis 310A. A pin 312A is slideably fittable to the opening 110A.

The pin 312A is slideably fittably to the pin guide opening 311A. The pin 312A may be self-drilling and self-tapping for insertion into the glenoid vault 116.

The opening 311 in the first sizing device 302A, is preferably normal or perpendicular to articulating surface 306A of the first sizing device 302A.

Continuing to refer to FIG. 9A, the kit 300A further includes a second sizing device 312A. The second sizing device 312A includes a support surface 314A and an opposed articulating surface 316A. An opening 317A is formed in the second sizing device 312A to provide pin guide 309A for the second sizing device 312A. The opening 317A is adapted for slideably fitting with the pin 312A. The opening 317A defines reconstructive axis 320A. The reconstructive axis 320 is preferably normal or perpendicular to the articulating surface 316A. Preferably, the reconstruction axis 320A is centrally positioned in the glenoid vault 116.

The second sizing device 312A includes a posterior protrusion 313 sufficiently different from the posterior protrusion 303A of the first sizing device 302A. As shown in FIG. 9A, for example, posterior protrusion 313A of the second sizing 312A is sufficiently larger than posterior protrusion, 303A of the first sizing device 302A.

The kit glenoid 300A further includes a first implant 322A. The first sizing device 302A is adapted for use with the first implant 322A. The first glenoid implant 322A therefore has a size and shape identical to the first sizing device 302A.

The kit 300A may further include a second glenoid implant 324A which has a size and shape identical to the second sizing device 312A. It should be appreciated that the first glenoid implant 322A and the second glenoid implant 324A do may include the openings of the first and second sizing device 302A and 312A, respectively.

The instruments 100, 200 as well as the sizing devices 302 and 312 may be made of any suitable, durable material. Preferably, the material for the instrument is sterilizable by common sterilizable techniques such as gamma irradiation, autoclaving or by other sterilizing techniques. The instruments of the present invention may be made of any suitable, durable material and may, for example, be made of a metal, a plastic, a ceramic or a composite. If made of a metal, the instrument 100, 200 and sizing devices 302 and 312 may be made of a cobalt chromium alloy, a stainless steel alloy, or a titanium alloy.

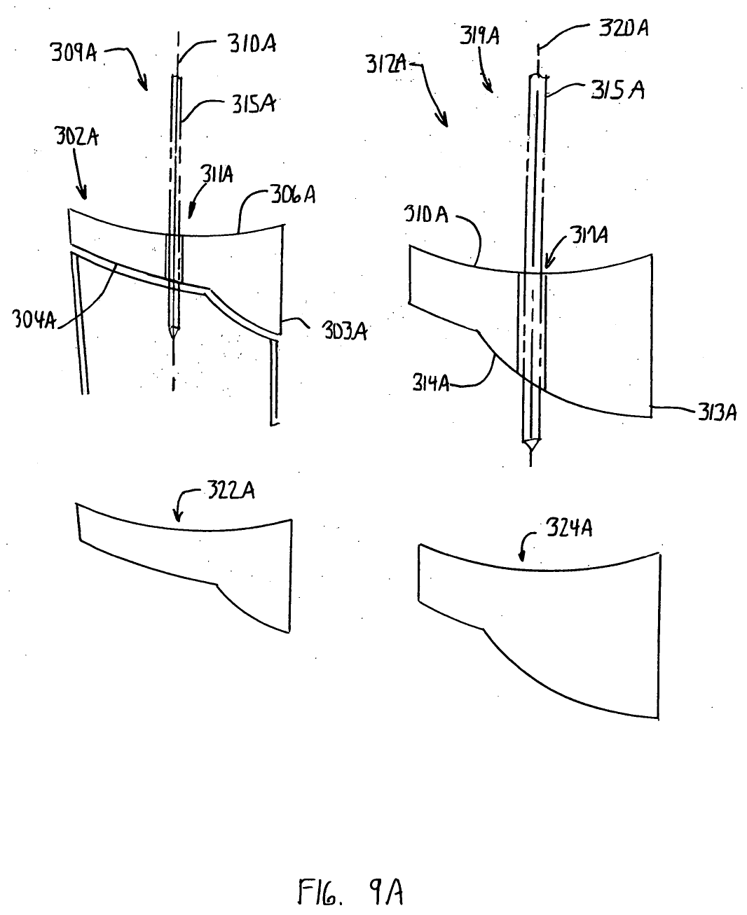

Referring now to FIG. 10 another embodiment of the present invention is shown as method 400 of performing shoulder arthoplasty. The method 400 includes a first step 402 of providing a first glenoid component for attachment to the glenoid the first glenoid component has a larger posterior dimension then the corresponding anterior dimension. The method 400 further includes a second step 404 of providing a second glenoid component for attachment to the glenoid. The second glenoid component has a larger posterior dimension than the corresponding anterior dimension and has one dimension different from that of the first glenoid component. The method 400 further includes a third step 406 of providing a first sizing device and a fourth step 408 of providing a second sizing device. A method 400 further includes a fifth step 410 of placing the first sizing device and a sixth step 402 of placing the second sizing against the glenoid fossa. The method 400 further includes a seventh step 414 of determining which of the first sizing device and the second sizing device should be implanted onto the scapula based on placing the one of the first sizing device and the second sizing device against the glenoid fossa. Method 400 further includes an eighth step 416 of implanting the proper of the first and second glenoid components onto the glenoid.

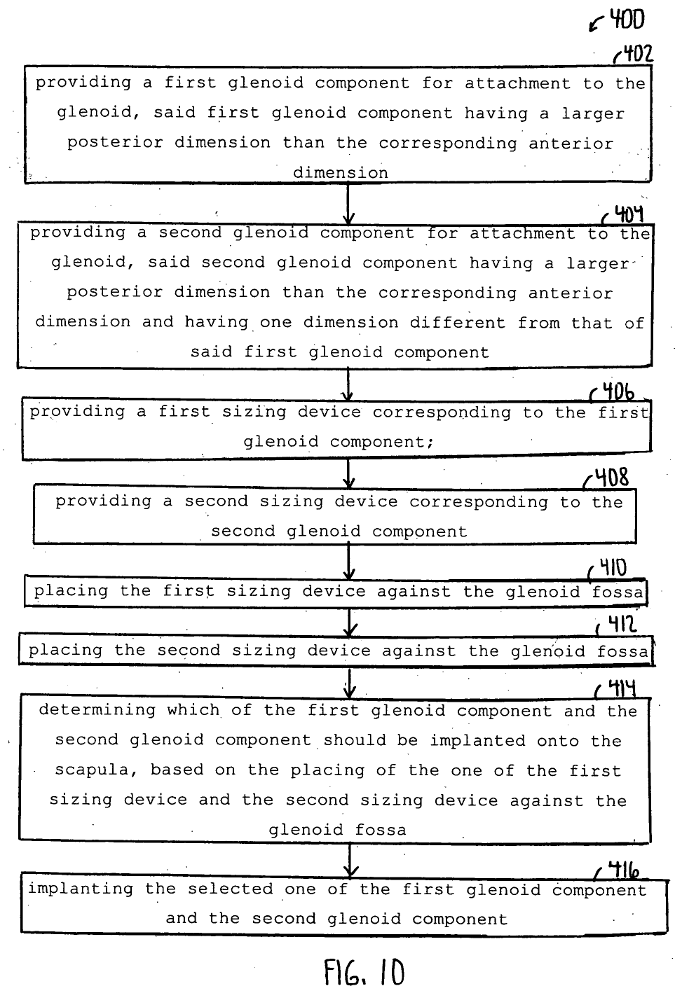

Referring now to FIG. 11, another embodiment of the present invention is shown as trial 502 which is part of instrument set 500. The trial 502 is similar to trial 302 of the instrument set 300 of FIG. 9.

For example and is shown in FIG. 11, the trial 502 includes a body 508. The body 508 defines a support surface 504 and an opposed articulating surface 506.

Unlike the trial 302 of the instrument 300 of FIG. 9, the trial 500 of the instrument set 500 includes a sensor 520 which may, as is shown in FIG. 11, be embedded or positioned in body 508 in the trial 502 and positioned below the articulating surface 506 of the trial 502. While the trial 502 of present invention may include a solitary sensor 520, the trial 502 may include an additional sensor 522 spaced from the first sensor 520. The sensors 520 and 522 may include an electrical conduits 524 for transmitting a signal 526 to controller 528 spaced from the trial 502.

The sensors 520 and 522 may be utilized to measure the joint loads and or the kinematics of the joint for which the trial and resulting prosthesis are to be used.

The sensors 520 and 522 may be sensors capable of measuring, for example, temperature, pressure, electrical current, or any other measurable characteristics at or around the articulating surface 506 of the trial 502. The sensors 520 and 522 may, for example, be pressure transducers. If pressure transducers 520 and 522 may, for example, be pressure transducers as shown in U.S. patent application Ser. No. 10/667,763_to Wasielewski incorporated herein reference is to entirety.

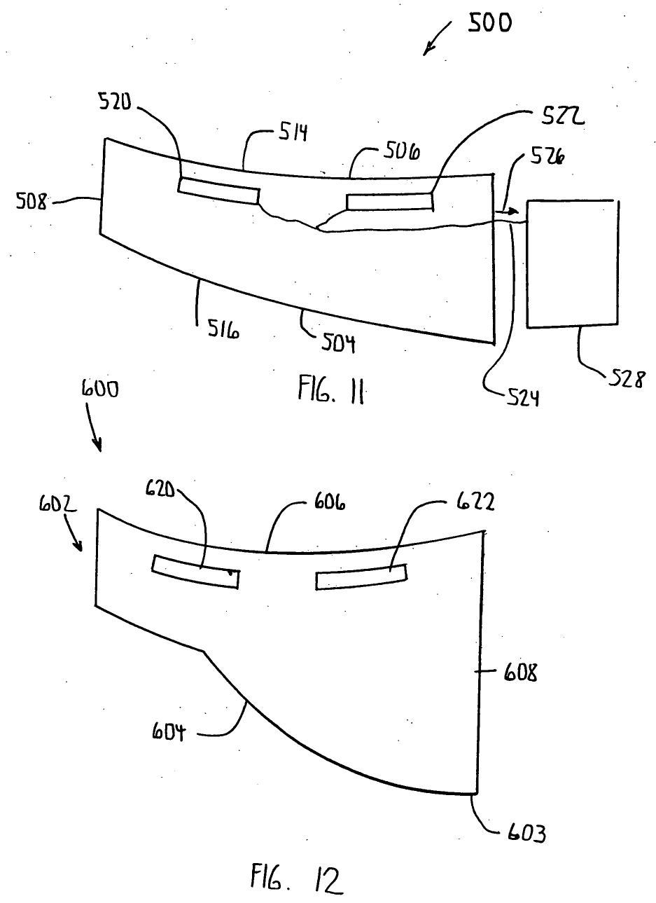

Referring now to FIG. 12, yet another embodiment of the present invention is shown as trial 602 for use with instrument set 600. The trial 602 includes a body 608. The body 608 includes a support surface 604 and a spaced apart articulating surface 606. Sensors 620 and 622 are embedded in the body 608 below the articulating surface 606 of the body 608 of the trial 602. The sensors 620 and 622 are similar to the sensors 520 and 522 of the trial 502 of FIG. 11. The body 608 of sensor 602 includes a posterior portion 603 for use with type C erosion or for posterior erosions.

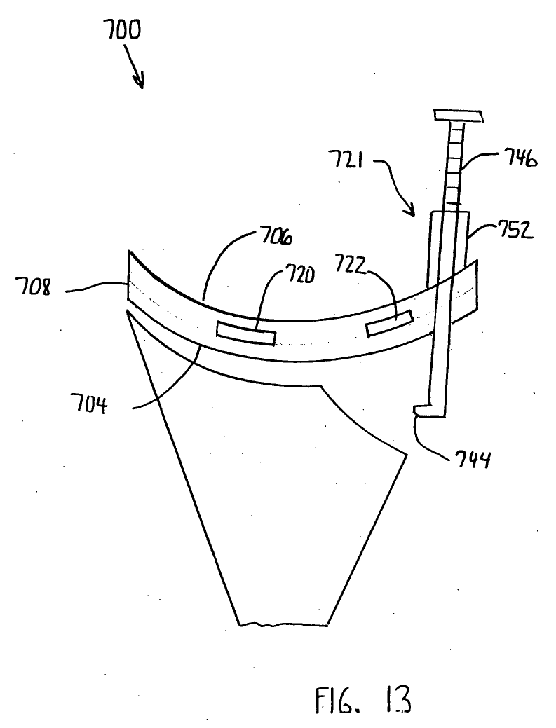

Referring now to FIG. 13, yet another embodiment of the present invention is shown as instrument 700. The instrument 700 includes a body 708 which defines a support surface 704 and an opposed articulating surface 706. The instrument 700 of FIG. 13 includes a probe 721 similar to the probe 720 of the instrument 10 of FIG. 8. The probe 721 includes a bushing 752 which may be secured to the body 708. The bushing 52 cooperates with stem 746 which is slideably fitted in the bushing 752. The stem 746 includes a contact area 744 which contacts the glenoid fossa at the posterior defect.

Instrument 700 further includes a first sensor 720 and second sensor 722 embedded in the instrument 700 and positioned below articulating surface 706 of the instrument 700. The sensors 720 and 722 are similar to the sensors 520 and 522 of the trial 502 of FIG. 11.

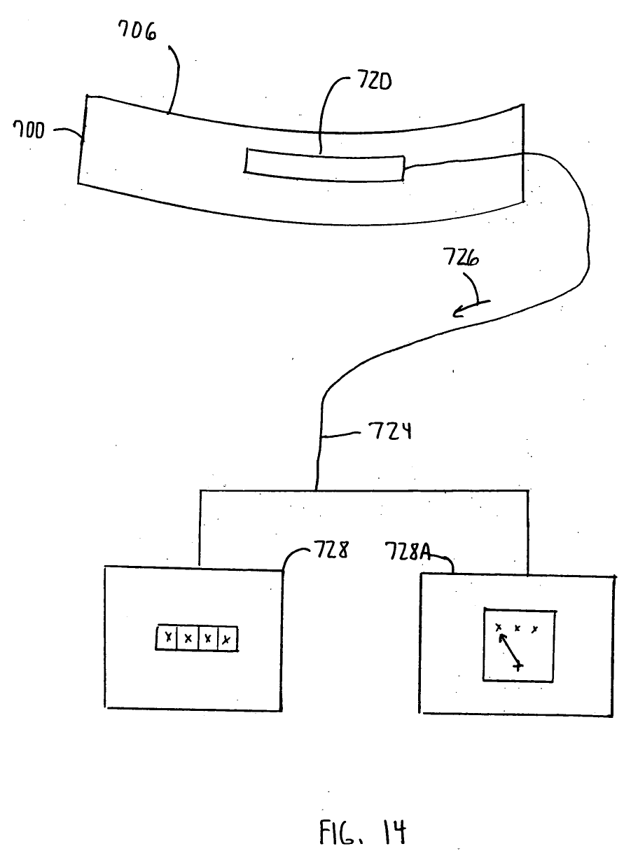

Referring now to FIG. 14, the sensor 720 of instrument 700 is shown in contact with transducer 728. A conduit is connected between sensor 720 and the transducer 728. The transducer 728 as shown in FIG. 14 may be in the form of a digital gage which can display digitally the feature measured by the sensor 520. For example, the transducer 728 may be in the form of a pressure gage being able to digitally display the pressure measured by the pressure sensor 720.

Alternatively and as is shown in FIG. 14, the gage 728 may be in the form of an analog gage 728A as shown in phantom. The analog gage 728A may include a meter that provides an analog measurement of the feature, for example, the pressure of as measured by the sensors 720.

Although the present invention and its advantages have been described in detail, it should be understood that various changes, substitutions, and alterations can be made therein without departing from the spirit and scope of the present invention as defined by the appended claims.

We claim:

1. An instrument for measuring a defect in a glenoid fossa of a scapula, comprising:
a member for contact with the glenoid fossa; and
a probe moveably associated with said member, said probe operably associated with the defect for measuring the defect in the scapula.

2. The instrument as in claim 1, wherein said member defines a convex surface for contact with the glenoid fossa.

3. The instrument as in claim 1:
wherein said member includes a wall defining an opening in said member; and
wherein said probe is slidably fitted to the opening.

4. The instrument as in claim 1, wherein said probe comprises indicia thereon for indicating the relative position of said probe with respect to said member.

5. The instrument as in claim 1, wherein said indicia comprise at least one of marks, shades, lines, colors, alphabetic characters or numeric characters.

6. The instrument as in claim 1, wherein said probe comprises a contact area for contact with the defect.

7. The instrument as in claim 6, wherein said contact area conforms to the shape of the defect.

8. The instrument as in claim 6, wherein said contact area is generally arcuate.

9. An instrument for measuring a defect in a glenoid fossa of a scapula, comprising:
a body adapted to be secured to the scapula; and
an element defining a surface thereof having a shape replicating that of a normal glenoid fossa, said element securable to said body.

10. The instrument as in claim 9, wherein said body defines a convex surface for contact with the glenoid fossa.

11. The instrument as in claim 9:
wherein said body comprises a protrusion for cooperation with an external cortical wall of the scapula, said protrusion adapted to secure the body to the scapula.

12. The instrument as in claim 9:
wherein said body extends from an end of said element.

13. The instrument as in claim 9:
wherein said body defines a longitudinal axis thereof;
wherein said element defines a longitudinal axis thereof; and
wherein the longitudinal axes define an included angle of about 30 to 100 degrees.

14. The instrument as in claim 9:
wherein said body comprises a protrusion for cooperation with an glenoid vault of the scapula, said protrusion adapted to secure the body to the scapula.

15. The instrument as in claim 9:
wherein said body defines an internal wall thereof, the wall defining a opening through said body; and
further comprising a pin for cooperation with an glenoid vault of the scapula, said pin adapted to be fitted to the internal wall of said body, said pin adapted to secure said body to the scapula.

16. A kit for measuring a defect in a worn glenoid fossa of a scapula, comprising:
a first sizing device defining a first surface for contact with the worn glenoid fossa and a second surface opposed to the first surface, the second surface having a shape conforming to a normal glenoid fossa, the first surface spaced from the second surface a first distance to represent a normal glenoid fossa; and
a second sizing device defining a first surface for contact with the worn glenoid fossa and a second surface opposed to the first surface, the second surface having a shape conforming to a normal glenoid fossa, the first surface spaced from the second surface a second distance to represent a normal glenoid fossa, the second distance and the first distance being different from each other.

17. The kit as in claim 16, further comprising:
a first glenoid implant corresponding to said first sizing device; and
a second glenoid implant corresponding to said second sizing device

18. The kit as in claim 16, wherein the first surface and the second surface of the said first sizing device are spaced apart by different dimension on the first end and the second end of the first sizing device.

19. A method for performing arthroplasty on a glenoid fossa of a scapula comprising the steps of:
providing a first glenoid component for attachment to the glenoid, said first glenoid component having a larger posterior dimension than the corresponding anterior dimension;
providing a second glenoid component for attachment to the glenoid, said second glenoid component having a larger posterior dimension than the corresponding anterior dimension and having one dimension different from that of said first glenoid component;
providing a first sizing device corresponding to the first glenoid component;
providing a second sizing device corresponding to the second glenoid component;
placing the first sizing device against the glenoid fossa;
placing the second sizing device against the glenoid fossa;
determining which of the first glenoid component and the second glenoid component should be implanted onto the scapula, based on the placing of the one of the first sizing device and the second sizing device against the glenoid fossa; and
implanting the selected one of the first glenoid component and the second glenoid component.

20. A method for performing arthroplasty on a glenoid as in claim 19, wherein the step of providing a sizing device step comprises the step of providing a sizing device having a body adapted to be secured to the scapula and an element defining a surface thereof having a shape replicating that of a normal glenoid fossa, the element securable to the body.

21. A method for performing arthroplasty on a glenoid as in claim 19, wherein the step of providing a sizing device comprises the step of providing a sizing device having a member for contact with the glenoid fossa and a probe moveably associated with the member, the probe operably associated with the defect for measuring the defect in the bone.

22. A method for performing arthroplasty on a glenoid as in claim 19:
wherein the step of providing a sizing device comprises the steps of providing a first sizing device defining a first surface for contact with the worn glenoid fossa and a second surface opposed to the first surface, the second surface having a shape conforming to a normal glenoid fossa, the first surface spaced from the second surface a first distance to represent a normal glenoid fossa; and
further comprising the step of providing a second sizing device defining a first surface for contact with the worn glenoid fossa and a second surface opposed to the first surface, the second surface having a shape conforming to a normal glenoid fossa, the first surface spaced from the second surface a second distance to represent a normal glenoid fossa, the second distance and the first distance being different from each other.
